(19) United States
(12) Patent Application Publication
(10) Pub. No.: US 2025/0375494 A1
(43) Pub. Date: Dec. 11, 2025

(54) FORMULA FOR TREATING ULCERATIVE COLITIS AND USE THEREOF

(71) Applicant: Qinghai University, Xining (CN)

(72) Inventors: Xiao Guo, Xining (CN); Qien Li, Xining (CN)

(21) Appl. No.: 19/054,791

(22) Filed: Feb. 15, 2025

(30) Foreign Application Priority Data
Jun. 6, 2024 (CN) 2024107285316

(51) Int. Cl. A61K 36/45; A61K 36/906; A61K 36/9062; A61P 1/00

(52) U.S. Cl. CPC A61K 36/45; A61K 36/906; A61K 36/9062; A61P 1/00; A61K 2236/333

(57) ABSTRACT

A formula for treating ulcerative colitis and a use thereof belong to the field of medical technologies. The formula includes Rhododendron anthopogonoides Maxim, Elettaria cardamomum Maton and Alpinia officinarum Hance. The formula, and an extract of the formula and a drug based on the formula can effectively restore an intestinal length of a body, protect integrity of intestinal barrier of a body, regulate imbalance of intestinal acid-base homeostasis of a body, inhibit death of intestinal epithelial cells of a body, and reduce a level of reactive oxygen species (ROS) in intestinal tract of a body, thereby achieving therapeutic effect on the ulcerative colitis.

### CROSS-REFERENCE TO RELATED APPLICATION

This application claims priority to Chinese Patent Application No. 202410728531.6, filed Jun. 6, 2024, which is herein incorporated by reference in its entirety.

### TECHNICAL FIELD

The disclosure relates to the field of medical technologies, and more particularly to a formula for treating ulcerative colitis and a use thereof.

### BACKGROUND

Ulcerative colitis (UC) is a chronic non-specific inflammatory disease of the colon and rectum, with lesions confined to the mucosa and submucosa of the large intestine. The lesions are mostly located in the sigmoid colon and rectum, and can also extend to the descending colon, even the whole colon. The main symptoms of UC include persistent diarrhea of varying severity, intermittent bloody stools, abdominal pain, and systemic symptoms, which may last for several weeks to several years. In addition, symptoms such as mucus bloody stools, abdominal distension, and fever may also occur.

At present, a most common treatment for UC is drug therapy, which includes glucocorticoids, aminosalicylic acids, immunosuppressants, biologics and others. However, many drugs are limited in clinical application. For example, long-term use of prednisone or dexamethasone to maintain treatment cannot prevent recurrence of UC, and may even bring a series of problems such as hepatorenal toxicity, drug dependence and recurrent disease after drug withdrawal. Some biologics are expensive and difficult for ordinary patients to afford, so it is urgent to seek new drugs with exact curative effect, less side effects and appropriate price for treating UC. With the development of traditional Chinese medicine and ethnic medicine in China, Tibetan medicine has shown great advantages in the prevention and treatment of chronic diseases, especially in the treatment of UC.

### SUMMARY

Embodiments of the disclosure develop a formula for treating ulcerative colitis. Extracts obtained by ethanol extraction and drugs based on the formula can treat the ulcerative colitis by restoring an intestinal length of a body, protecting integrity of intestinal barrier of a body, regulating imbalance of intestinal acid-base homeostasis of a body, inhibiting death of intestinal epithelial cells of a body, and reducing a level of reactive oxygen species (ROS) in intestinal tract of a body.

In order to achieve the above purposes, embodiments of the disclosure can adopt the following technical solutions.

In a first aspect, the disclosure provides a formula for treating ulcerative colitis. The formula includes Rhododendron anthopogonoides Maxim, Elettaria cardamomum Maton and Alpinia officinarum Hance, and thus the formula hereinafter also is referred to as three-ingredient Rhododendron formula. It should be noted that, according to Tibetan medical theory, the three-ingredient Rhododendron formula not only has functions of soothing liver, strengthening stomach, and strengthening heart, but also has a good therapeutic effect on the ulcerative colitis. In addition, the formula is simple, and two of three medicinal materials are food-medicine homologous materials, which have good medicinal properties.

In an embodiment, a weight ratio of the Rhododendron anthopogonoides Maxim:the Elettaria cardamomum Maton:the Alpinia officinarum Hance is 2:1:1, 1:1:1, or 20:15:8.

It should be noted that the weight ratio of the Rhododendron anthopogonoides Maxim:the Elettaria cardamomum Maton:the Alpinia officinarum Hance can be adjusted according to a specific situation. In a specific embodiment of the disclosure, the weight ratio is 2:1:1, and an extract obtained by ethanol extraction at this weight ratio has better therapeutic effects than extracts obtained by ethanol extraction at other weight ratios.

In a second aspect, the disclosure provides an extract of the formula for treating the ulcerative colitis. The extract of the formula is extracted from the formula with ethanol or water.

It should be noted that, the formula is extracted with the ethanol for subsequent medicinal use, and an extraction method can be carried out according to methods known in the art.

In an embodiment, a preparation method of the extract of the formula includes one of the following methods: (1) crushing the Rhododendron anthopogonoides Maxim, the Elettaria cardamomum Maton and the Alpinia officinarum Hance, and then performing hot reflux extraction with the ethanol, followed by centrifuging, rotary evaporating, and freeze-drying to thereby obtain the extract of the formula, where the weight ratio of the Rhododendron anthopogonoides Maxim:the Elettaria cardamomum Maton:the Alpinia officinarum Hance is 2:1:1; (2) crushing the Rhododendron anthopogonoides Maxim, the Elettaria cardamomum Maton and the Alpinia officinarum Hance, and then performing hot reflux extraction with the water, followed by centrifuging, rotary evaporating, and freeze-drying to thereby obtain the extract of the formula, where the weight ratio of the Rhododendron anthopogonoides Maxim:the Elettaria cardamomum Maton:the Alpinia officinarum Hance is 2:1:1, 1:1:1 or 20:15:8.

It should be noted that, the formula extracted with the ethanol can be carried out using the methods known in the art. In a specific embodiment of the disclosure, the above preparation method is used, and the extract of the formula obtained by the above preparation method has better therapeutic effects than other methods. In the specific embodiment, the preparation method of the extract of the formula includes: crushing the Rhododendron anthopogonoides Maxim, the Elettaria cardamomum Maton and the Alpinia officinarum Hance into 80 meshes, performing the hot reflux extraction twice with the water or 80% ethanol (i.e., the ethanol with a volume fraction of 80%), and then combining filtrates, followed by centrifuging, rotary evaporating, and freeze-drying to obtain the extract of the formula.

In a third aspect, the disclosure provides a drug for treating the ulcerative colitis, which includes the extract of the formula.

It should be noted that, the extract of the formula can be prepared into the drug for treating the ulcerative colitis. Of course, the extract of the formula can also be used directly as the drug.

In an embodiment, the drug can further include other effective active ingredients for treating or improving the ulcerative colitis.

It should be noted that, in order to increase the therapeutic effects, the extract of the formula of the disclosure can be combined with the other effective active ingredients that can treat or improve the ulcerative colitis to prepare the drug for treating the ulcerative colitis. It should be understood that, the other effective active ingredients that can treat or improve the ulcerative colitis are well-known in the art, such as 5-aminosalicylic acid drugs, rhubarb, aloe, senna, and other traditional Chinese medicines or active components like Chinese medicine polyphenols, alkaloids, quinones, terpenes, etc.

In an embodiment, the drug can further include a pharmaceutically acceptable carrier.

It should be noted that, in order to prepare the drug into different dosage forms, the pharmaceutically acceptable carrier can be added for preparation. The pharmaceutical carrier is adapted for powders, granules, tablets, pills, capsules, oral liquids, or ointments. In addition, those skilled in the art can select a pharmaceutically acceptable carrier according to a specific dosage form.

In a fourth aspect, the disclosure provides a use of the formula for treating the ulcerative colitis, or the extract of the formula in preparation of the drug for treating the ulcerative colitis.

In an embodiment, the use is one or more of the group of (1) through (5) as follows: (1) a use of the extract of the formula in preparation of the drug for restoring an intestinal length of a body; (2) a use of the extract of the formula in preparation of the drug for protecting integrity of intestinal barrier of a body; (3) a use of the extract of the formula in preparation of the drug for regulating imbalance of intestinal acid-base homeostasis of a body; (4) a use of the extract of the formula in preparation of the drug for inhibiting death of intestinal epithelial cells of a body; and (5) a use of the extract of the formula in preparation of the drug for reducing a level of reactive oxygen species (ROS) in intestinal tract of a body.

In an embodiment, dosage forms of the drug in the use can include powders, granules, tablets, pills, capsules, oral liquids, or ointments.

The beneficial effects of the disclosure at least include: the formula, the extract of the formula and the drug for treating the ulcerative colitis provided by the disclosure can effectively restore the intestinal length of the body, protect the integrity of the intestinal barrier of the body, regulate the imbalance of the intestinal acid-base homeostasis of the body, inhibit the death of the intestinal epithelial cells of the body and reduce the level of ROS in the intestinal tract of the body, thereby achieving the purpose of treating the ulcerative colitis.

### BRIEF DESCRIPTION OF DRAWINGS

FIG. 1 illustrates effect of an extract of 2:1:1 three-ingredient Rhododendron formula by ethanol extraction in Tibetan medicine on cell viability of rat lung epithelial cell line-6 t-antigen negative (RLE-6TN) cells, where n=6. Compared to a normal control group, **p<0.01, *p<0.05.

FIG. 2 illustrates effect of an extract of 1:1:1 three-ingredient Rhododendron formula by water extraction on the cell viability of the RLE-6TN cells, where n=6. Compared to the normal control group, **p<0.01, *p<0.05.

FIG. 3 illustrates effect of an extract of 2:1:1 three-ingredient Rhododendron formula by water extraction on the cell viability of the RLE-6TN cells, where n=6. Compared to the normal control group, **p<0.01, *p<0.05.

FIG. 4 illustrates effect of an extract of 20:15:8 three-ingredient Rhododendron formula by water extraction on the cell viability of the RLE-6TN cells, where n=6. Compared to the normal control group, **p<0.01, *p<0.05.

FIG. 5 illustrates a survival rate of female fruit flies (also referred as to Percent survival-Female) of each group.

FIG. 6 illustrates a survival rate of male fruit flies (also referred as to Percent survival-Male) of each group.

FIG. 7 illustrates a motor ability of fruit flies in each group.

FIG. 8 illustrates an excretory capacity of the fruit flies in each group.

FIG. 9 illustrates an actual fruit fly after a “Smurf” experiment.

FIG. 10 illustrates a percentage of “Smurf” fruit flies obtained from the “Smurf” experiment.

FIG. 11 illustrates intestinal lengths of fruit flies after modeling with different concentrations of dextran sulfate sodium (DSS) for 72 hours.

FIG. 12 illustrates different imbalances of intestinal acid-base homeostasis in UC fruit flies.

FIG. 13 illustrates proportions of the UC fruit flies with different imbalances of the intestinal acid-base homeostasis regulated by different concentrations of the extract of three-ingredient Rhododendron formula by ethanol extraction.

FIG. 14 illustrates improvement of intestinal cells treated with the extract of three-ingredient Rhododendron formula by ethanol extraction that is labeled with trypan blue dye.

FIG. 15 illustrates improvement of the intestinal cells treated with different concentrations of the extract of three-ingredient Rhododendron formula by ethanol extraction.

FIG. 16 illustrates fluorescence-labeled intestinal epithelial cells of the fruit flies after administration of the extract of three-ingredient Rhododendron formula by ethanol extraction.

FIG. 17 illustrates fluorescence intensity of different experimental groups.

FIG. 18 illustrates fluorescence labeled with dihydroethidium (DHE) dye in the different experimental groups.

FIG. 19 illustrates intestinal oxidative damage in the UC fruit flies across the different experimental groups.

### DETAILED DESCRIPTION OF EMBODIMENTS

The embodiments are intended to better illustrate the disclosure, but the scope of the disclosure is not limited to the embodiments. Therefore, any non-essential modifications and adjustments to the implementation methods made by those skilled in the art based on the above-described content of the disclosure still fall within the scope of protection of the disclosure.

The terms used herein are used only to describe specific embodiments and are not intended to limit the disclosure. Expressions in the singular include expressions in the plural unless they have a significantly different meaning in the context. As used herein, it should be understood that terms such as “include”, “have”, “comprise” are intended to indicate the presence of features, numbers, operations, components, parts, elements, materials, or combinations. The terms of the disclosure are disclosed in the specification and is not intended to exclude the possibility that one or more other features, numbers, operations, components, components, elements, materials or combinations thereof may exist or may be added. As used here, “/” can be interpreted as “and” or “or”.

In order to better understand the disclosure, the content of the disclosure is further illustrated in combination with specific embodiments, but the content of the disclosure is not limited to the following embodiments.

First, preparation of an extract of three-ingredient Rhododendron formula

### Embodiment 1 Preparation of an Extract of 2:1:1 Three-Ingredient Rhododendron Formula by Ethanol Extraction

Rhododendron anthopogonoides Maxim, Elettaria cardamomum Maton and Alpinia officinarum Hance are mixed in a weight ratio of 2:1:1 and crushed into 80 meshes, and hot reflux extraction with 80% ethanol is performed twice. Then filtration is performed using a filter with a pore size of 0.45 micrometers (μm) to obtain filtrates, and the filtrates are combined and centrifuged at 5,000 radians per minute (rad/min) to obtain supernatant. The supernatant is subjected to reduced pressure rotary evaporation at 45° C. and −0.075 Megapascal (MPa) to obtain a concentrated liquid, and the concentrated liquid is stored at −80° C. for 72 hours. The extract of 2:1:1 three-ingredient Rhododendron formula by ethanol extraction is obtained by freeze drying the concentrated liquid at −86.8° C. and 8 Pascal (Pa) for 60 hours.

### Embodiment 2 Preparation of an Extract of 1:1:1 Three-Ingredient Rhododendron Formula by Water Extraction

Rhododendron anthopogonoides Maxim, Elettaria cardamomum Maton and Alpinia officinarum Hance are mixed in a weight ratio of 1:1:1 and grinded into 80 meshes, and hot reflux extraction with distilled water is performed twice. Then filtration is performed using a filter with a pore size of 0.45 μm to obtain filtrates, and the filtrates are combined and centrifuged at 5,000 rad/min to obtain supernatant. The supernatant is subjected to reduced pressure rotary evaporation at 60° C. and −0.075 MPa to obtain a concentrated liquid, and the concentrated liquid is stored at −80° C. for 72 hours. The extract of 1:1:1 three-ingredient Rhododendron formula by water extraction is obtained by freeze drying the concentrated liquid at −86.8° C. and 8 Pa for 60 hours.

### Embodiment 3 Preparation of an Extract of 2:1:1 Three-Ingredient Rhododendron Formula by Water Extraction

Rhododendron anthopogonoides Maxim, Elettaria cardamomum Maton and Alpinia officinarum Hance are mixed in a weight ratio of 2:1:1 and grinded into 80 meshes, and hot reflux extraction with distilled water is performed twice. Then filtration is performed using a filter with a pore size of 0.45 μm to obtain filtrates, and the filtrates are combined and centrifuged at 5,000 rad/min to obtain supernatant. The supernatant is subjected to reduced pressure rotary evaporation at 60° C. and −0.075 MPa to obtain a concentrated liquid, and the concentrated liquid is stored at −80° C. for 72 hours. The extract of 2:1:1 three-ingredient Rhododendron formula by water extraction is obtained by freeze drying the concentrated liquid at −86.8° C. and 8 Pa for 60 hours.

### Embodiment 4 Preparation of an Extract of 20:15:8 Three-Ingredient Rhododendron Formula by Water Extraction

Rhododendron anthopogonoides Maxim, Elettaria cardamomum Maton and Alpinia officinarum Hance are mixed in a weight ratio of 20:15:8 and grinded into 80 meshes, and hot reflux extraction with distilled water is performed twice. Then filtration is performed using a filter with a pore size of 0.45 μm filter to obtain filtrates, and the filtrates are combined and centrifuged at 5,000 rad/min to obtain supernatant. The supernatant is subjected to reduced pressure rotary evaporation at 60° C. and −0.075 MPa to obtain a concentrated liquid, and the concentrated liquid is stored at −80° C. for 72 hours. The extract of 20:15:8 three-ingredient Rhododendron formula by water extraction is obtained by freeze drying the concentrated liquid at −86.8° C. and 8 Pa for 60 hours.

Second, toxicity experiments of medicated intestinal absorbent fluids from different formula ratios on RLE-6TN cells

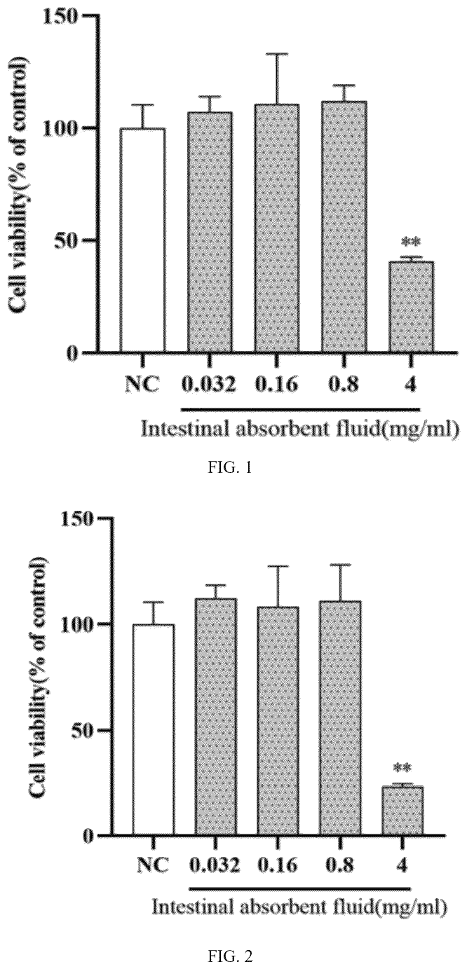

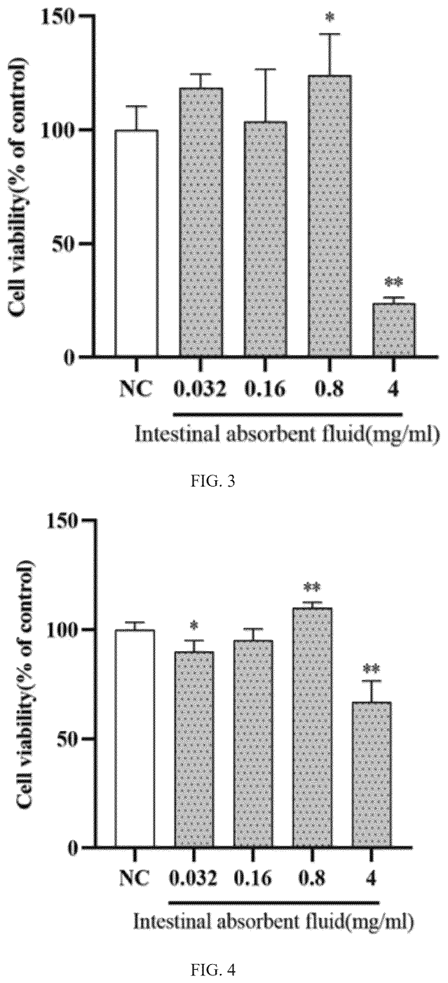

The extract of 2:1:1 three-ingredient Rhododendron formula by ethanol extraction, the extract of 1:1:1 three-ingredient Rhododendron formula by water extraction, the extract of 2:1:1 three-ingredient Rhododendron formula by water extraction, and the extract of 20:15:8 three-ingredient Rhododendron formula by water extraction are respectively formulated into solutions with concentrations of 0.032 milligrams per milliliter (mg/mL), 0.16 mg/mL, 0.8 mg/mL, and 4 mg/mL. These extracts at different concentrations and from the different formula ratios are added to culture of RLE-6TN cells for 24 hours. The cell viability of the RLE-6TN cells is then determined using a cell counting kit-8 (CCK-8) test kit, and impact of the extracts from the different formula ratios on the cell viability of the RLE-6TN cells is evaluated by repeating each extract for 6 times at each concentration. As shown in FIGS. 1 to 4, a result indicates that, at a concentration of 0.8 mg/mL, none of the extracts from the different formula ratios shows significant toxicity to the RLE-6TN cells. The extract of 2:1:1 three-ingredient Rhododendron formula by ethanol extraction has a best toxicity.

Third, comparative study of efficacy of the medicated intestinal absorbent fluids from the different formula ratios of three-ingredient Rhododendron formula in treating ulcerative colitis

Half-maximal effective concentrations (EC₅₀) of the extract of 2:1:1 three-ingredient Rhododendron formula by ethanol extraction, the extract of 1:1:1 three-ingredient Rhododendron formula by water extraction, the extract of 2:1:1 three-ingredient Rhododendron formula by water extraction, and the extract of 20:15:8 three-ingredient Rhododendron formula by water extraction are respectively tested. It is found that, the medicated intestinal absorbent fluids from the different formula ratios of three-ingredient Rhododendron formula all show good efficacy in treating the ulcerative colitis. The extract of 2:1:1 three-ingredient Rhododendron formula by ethanol extraction has a highest EC₅₀, indicating best safety and effectiveness, and a result is shown in Table 1.

TABLE 1
the EC₅₀ of the extracts of the formula
from the different formula ratios

| three-ingredient Rhododendron formula medicated | |
|---|---|
| intestinal absorbent solution | EC₅₀ (mg/mL) |
| extract of 1:1:1 three-ingredient Rhododendron formula | 0.4232 |
| by water extraction | |
| extract of 20:15:8 three-ingredient Rhododendron | 0.5196 |
| formula by water extraction | |
| extract of 2:1:1 three-ingredient Rhododendron formula | 0.4065 |
| by water extraction | |
| extract of 2:1:1 three-ingredient Rhododendron formula | 0.5385 |
| by ethanol extraction | |

Fourth, experiments on protective effects of extract of three-ingredient Rhododendron formula on physiological function damage caused by a fruit fly UC model.

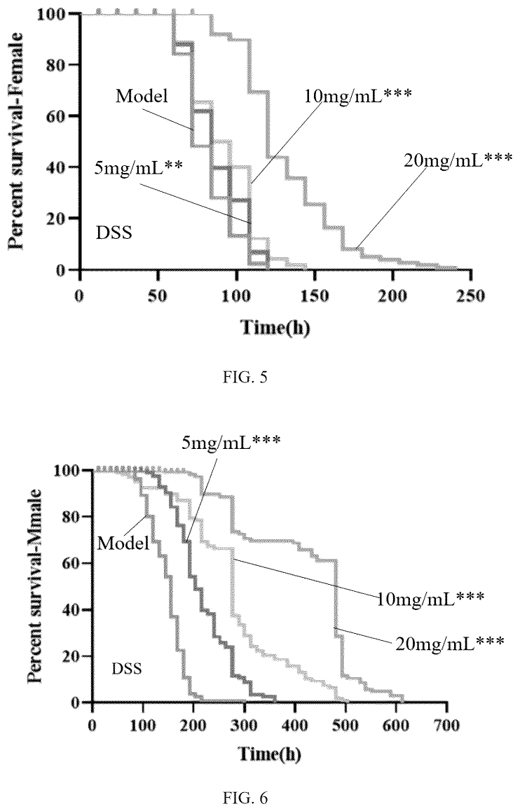

In an embodiment of the disclosure, in order to determine the anti-inflammatory effect of the extract of 2:1:1 three-ingredient Rhododendron formula by ethanol extraction (hereinafter referred to as the extract of three-ingredient Rhododendron formula by ethanol extraction), a survival rate of male and female fruit flies stimulated by dextran sodium sulfate (DSS) is measured. Specific steps are as follows: w¹¹¹⁸ strain fruit flies that have eclosed for 2-4 days in a common medium and a medicated medium are collected randomly. There are 20 fruit flies per bottle, and they are starved for 30 minutes. The fruit flies in different treatment groups are transferred to a filter paper culture medium dripping with 4% DSS (5% sucrose), and a number of dead fruit flies is recorded every 6 hours or 12 hours. The filter paper is changed every 72 hours, and each group is repeated with a total of 9 bottles. As shown in FIG. 5 and FIG. 6, a result show that, the extract of three-ingredient Rhododendron formula by ethanol extraction significantly prolongs a survival rate of UC fruit flies induced by the DSS, indicating that the extract of three-ingredient Rhododendron formula by ethanol extraction has significant anti-inflammatory effect.

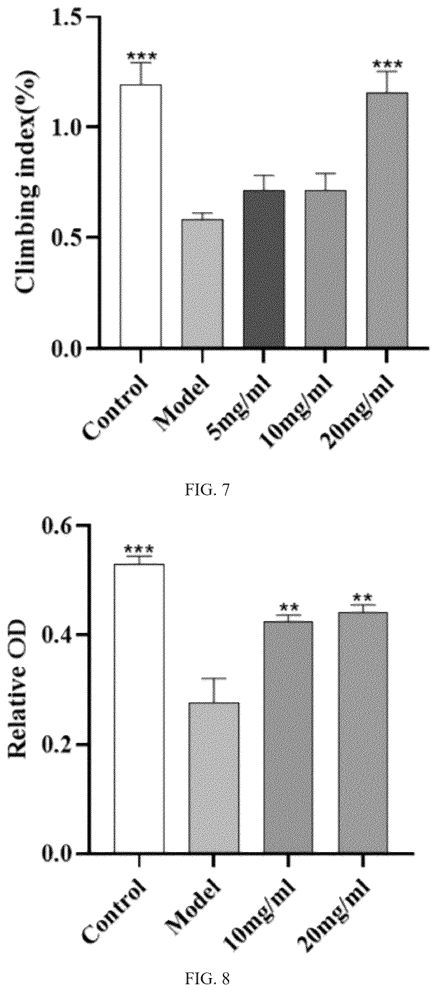

In the embodiment of the disclosure, an effect of the extract of three-ingredient Rhododendron formula by ethanol extraction on an overall physiological function of the UC fruit flies induced by the DSS is further determined by measuring a motor ability and an excretion capacity of the fruit flies. An operation of measuring the motor ability is as follows: 20 fruit flies that have eclosed for 3 days in a common medium and a medicated medium are collected randomly and fed with a 4% DSS (a 5% sucrose) solution or without the 4% DSS (the 5% sucrose) solution for 24 hours. The fruit flies are shaken gently to a bottom of a test tube, and a bottle cap is plugged. a number of fruit flies climbing more than 8 centimeters (cm) in 5 seconds(s) is recorded, and the experiment is repeated for 5 times in an environment of 25° C. and 60% humidity. An operation of measuring the excretion capacity is as follows: 20 fruit flies that have eclosed for 3 days in a common medium and a medicated medium are collected randomly to an agar medium and starved for 6 hours, and there are 20 fruit flies in each group. Then the fruit flies are placed in 2 milliliters (mL) centrifuge tubes containing 200 microliters (μL) of 5% sucrose and 2% bromophenol blue reagent, respectively, and fed for 12 hours. After feeding, the 2 mL centrifuge tube is placed in a 5 mL centrifuge tube for vibration, 150 μL of 5% sucrose and 2% bromophenol blue reagent is put in a 96 well plate to measure optical density at 625 nm (OD₆₂₅). As shown in FIG. 7 and FIG. 8, a result show that, compared to a model group, the extract of three-ingredient Rhododendron formula by ethanol extraction can improve the motor ability and the excretion capacity of the UC fruit flies. Therefore, the extract of three-ingredient Rhododendron formula by ethanol extraction has a protective effect on the physiological function damage caused by the fruit fly UC model.

Fifth, experiments on improvement of intestinal morphology and acid-base imbalance in the UC fruit flies by the extract of the three-ingredient Rhododendron formula by ethanol extraction

Female w¹¹¹⁸ fruit flies that have eclosed for 2-4 days are collected randomly, and are fed in a common medium and a drug-containing medium for 7 days. After 72 hours of modeling with 4% DSS (i.e., using 4% DSS to induce ulcerative colitis), corresponding groups of the female fruit flies are collected to an agar medium and starved for 6 hours. (1) The female fruit flies are placed in a tube containing 5% sucrose and 2% bromophenol blue reagent and are fed for 18 hours. Under a microscope, a number of “Smurfs” is selected and calculated. (2) 15-20 complete intestinal tracts of the female fruit flies are separated under the microscope and placed in phosphate buffered saline (PBS), and intestinal morphological changes are observed under the microscope, and intestinal lengths are measured with a vernier caliper. (3) The female fruit flies are placed in a fruit fly tube containing 5% sucrose and 4% pH indicator reagent and fed for 4 hours. 15-20 complete fruit fly intestinal tracts are separated under the microscope, and copper cell regions are observed. The above experiments are repeated three times per group.

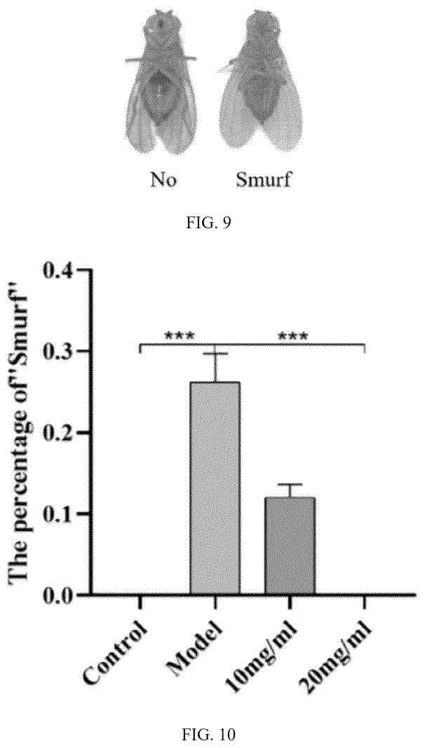

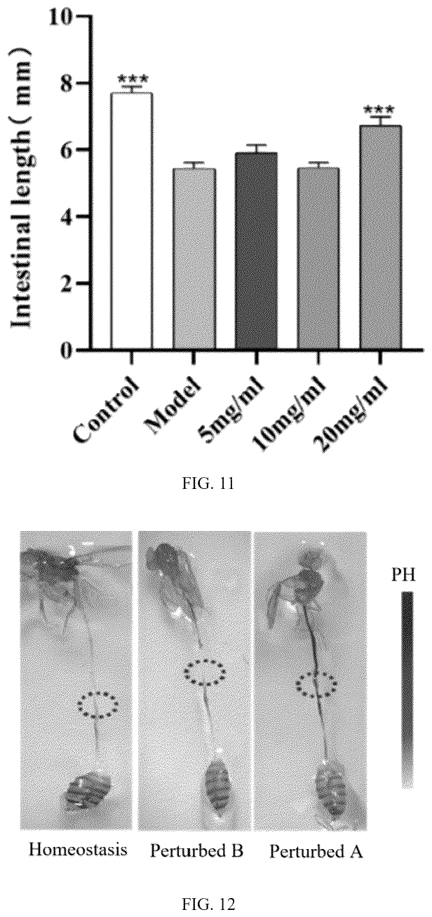

A “Smurf” experiment is used to evaluate the integrity of the intestinal barrier. A result shows that obvious “Smurf” phenomenon is appeared in the fruit flies after modeling with the 4% DSS for 72 hours, indicating that the intestinal barrier is damaged. The extract of three-ingredient Rhododendron formula by ethanol extraction has a protective effect on the intestinal barrier of the fruit flies (as shown in FIG. 9 and FIG. 10). After 72 hours of modeling with the 4% DSS, the fruit fly intestinal lengths are significantly shortened, and the fruit fly intestinal lengths are significantly restored after administration of the extract of three-ingredient Rhododendron formula by ethanol extraction (as shown in FIG. 11).

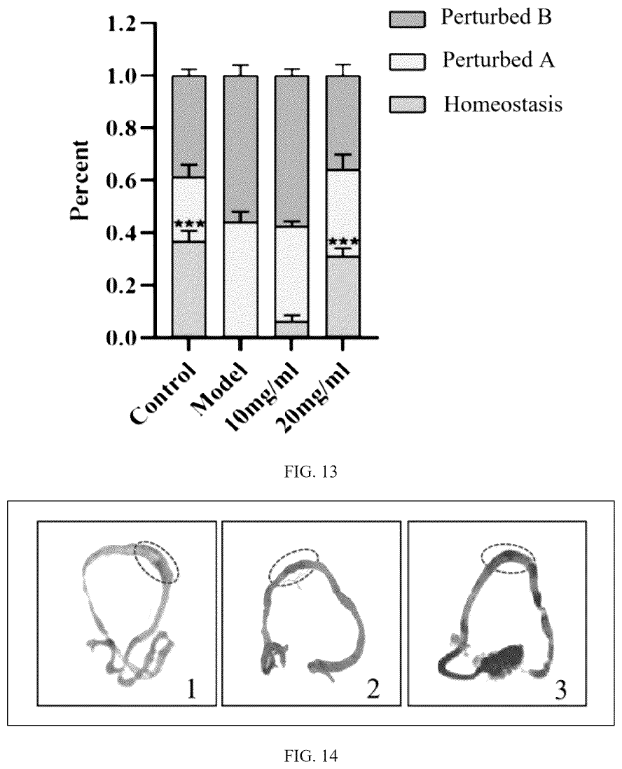

In addition, a fruit fly intestinal pH test shows that, after administration of the extract of three-ingredient Rhododendron formula by ethanol extraction, can regulate the imbalance of the intestinal acid-base homeostasis in the UC fruit flies can be regulated (as shown in FIG. 12 and FIG. 13).

In conclusion, the extract of three-ingredient Rhododendron formula by ethanol extraction can restore the intestinal lengths of the fruit flies, protect the integrity of intestinal barrier and regulate the imbalance of the acid-base homeostasis.

Sixth, experiments on inhibition of death of intestinal epithelial cells in the fruit flies by the extract of three-ingredient Rhododendron formula by ethanol extraction

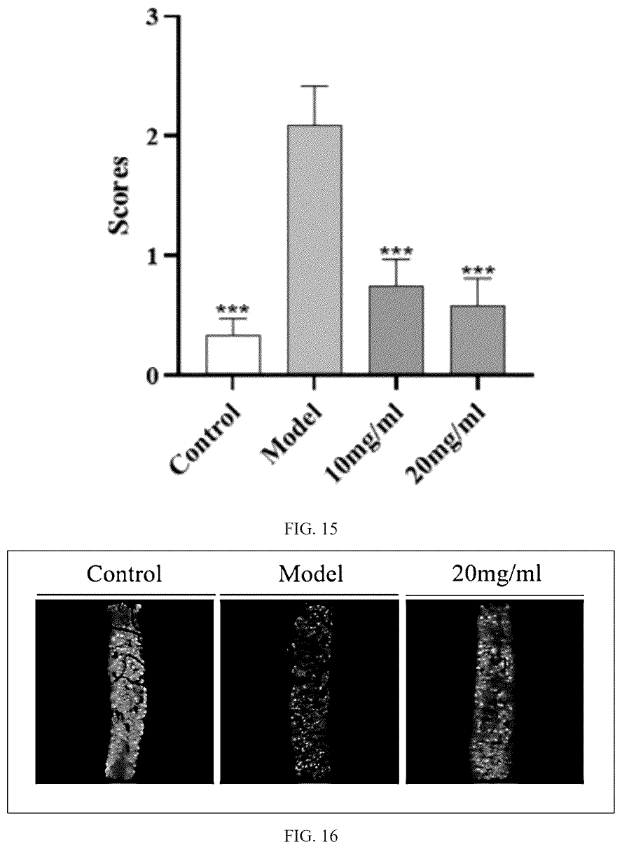

When the UC occurs, programmed cell death of the intestinal epithelial cells increases, thereby disrupting the intestinal barrier. Fruit flies that have eclosed for 2-4 days are collected randomly, and after 2 days of recovery, the fruit flies are starved for 2 hours, followed by modeling with 4% DSS for 72 hours. Afterward, the fruit flies are transferred to food containing 0.4% trypan blue and are fed for 4 hours. 15-20 complete fruit fly intestinal tracts are separated and observed under a microscope. Each group of the experiment is repeated three times. The trypan blue dye solution is used to label dead intestinal cells. After modeling with the 4% DSS for 72 hours, the blue degree of fruit fly midgut significantly increases, indicating that the DSS can cause the death of the intestinal epithelial cells. After administration of the extract of three-ingredient Rhododendron formula by ethanol extraction, the degree of the death of the intestinal epithelial cells are improved. (as shown in FIG. 14 and FIG. 15).

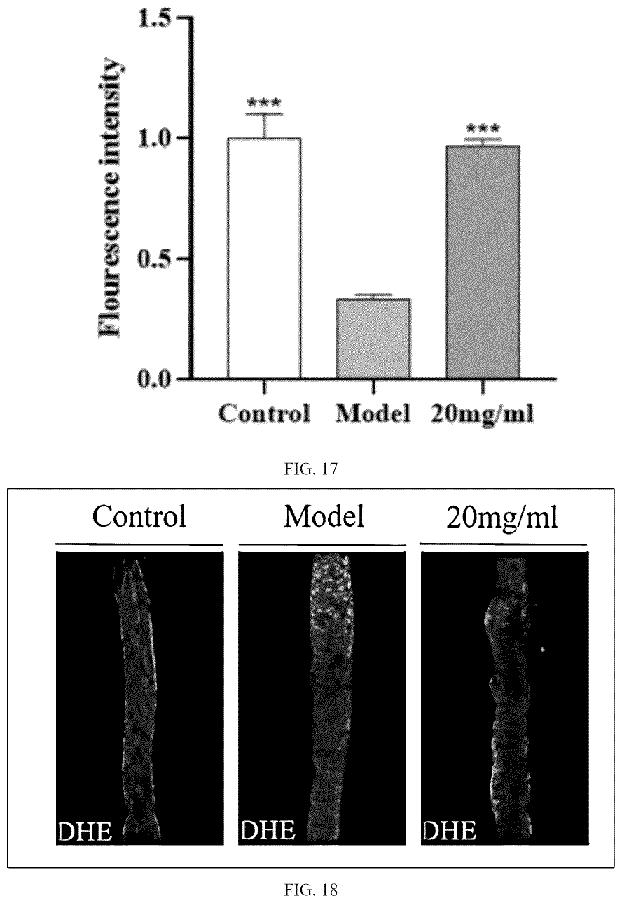

In order to further verify the death of the fruit fly intestinal epithelial cells, Myo1A-Gal4; UAS-GFP strain fruit flies that have eclosed for 2-4 days are collected randomly. After 2 days of recovery, the Myo1A-Gal4; UAS-GFP strain fruit flies are starved for 2 hours, followed by modeling with 4% DSS for 72 hours. Under the microscope, 15-20 complete fruit fly intestinal tracts are separated and placed in 4% paraformaldehyde. The 15-20 complete fruit fly intestinal tracts are fixed at room temperature for 30 minutes, washed three times with PBS for 5 minutes each, stained with DAPI for 10 minutes, washed three times with PBS for 5 minutes each, and observed and photographed under a fluorescence microscope after glycerol sealing. Each group of the experiment is repeated three times. Using the Myo1A-Gal4; UAS-GFP transgenic fruit flies to mark the intestinal epithelial cells, after modeling with the 4% DSS for 72 hours and administration of the extract of three-ingredient Rhododendron formula by ethanol extraction, the fluorescence intensity in the intestinal tracts is significantly enhanced compared to a model group, indicating that the extract of three-ingredient Rhododendron formula by ethanol extraction can inhibit the death of the fruit fly intestinal epithelial cells (FIGS. 16 and 17).

Seventh, experiments on improvement of intestinal oxidative damage in fruit flies by the extract of three-ingredient Rhododendron formula by ethanol extraction

The UC is commonly associated with overproduction of ROS. A content of the ROS in intestinal tract can be measured using a peroxide indicator dihydroethidium (DHE) dye. A specific procedure is as follows: fruit flies that have eclosed for 2-4 days from a normal culture medium are randomly collected, allow them to recover for 2 days, then starve them for 2 hours, followed by modeling with 4% DSS for 72 hours. Under a microscope, 15-20 complete fruit fly intestinal tracts are separated and placed in PBS. The 15-20 complete fruit fly intestinal tracts are stained with 1:200 diluted DHE at room temperature for 30 minutes, followed by washing with PBS three times, 5 minutes each. The 15-20 complete fruit fly intestinal tracts are then fixed with 4% formaldehyde for 30 minutes, washed with PBS three times, 5 minutes each, and stained with 4′,6-diamidino-2-phenylindole (DAPI) for 10 minutes. Finally, the 15-20 complete fruit fly intestinal tracts are washed with PBS three times, 5 minutes each. After sealing with glycerin, the 15-20 complete fruit fly intestinal tracts are observed under a fluorescence microscope. Each experiment is repeated three times.

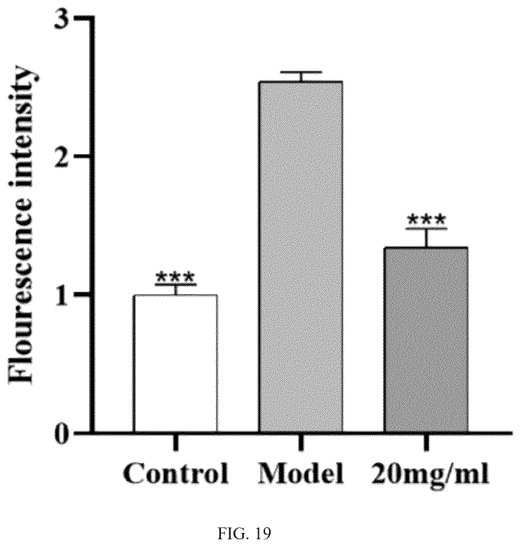

AS shown in FIGS. 18 and 19, a result indicates that after modeling with the 4% DSS for 48 hours, the intensity of the DHE in the intestinal tract is significantly increased. After administration of the extract of the three-ingredient Rhododendron formula by ethanol extraction, the intensity of the DHE in the intestinal tract decreases, indicating that the extract of three-ingredient Rhododendron formula by ethanol extraction can reduce the level of ROS in the fruit fly intestinal tract.

It should be noted that the above embodiments are provided to illustrate the technical solutions of the disclosure and are not intended to be limiting. Although the disclosure has been described in detail with reference to specific embodiments, those skilled in the art should understand that modifications or equivalent substitutions can be made to the technical solutions of the disclosure without departing from the spirit and scope of the disclosure. All such modifications and substitutions are intended to be covered by the claims of the disclosure.

1-6. (canceled)

7. A drug for treating ulcerative colitis, wherein the drug comprises a formula extract of Rhododendron anthopogonoides Maxim, Elettaria cardamomum Maton, and Alpinia officinarum Hance; a weight ratio of the Rhododendron anthopogonoides Maxim:the Elettaria cardamomum Maton:the Alpinia officinarum Hance is 2:1:1, and the formula extract is ethanolic extract obtained by ethanol extraction with 80% ethanol.

8. The drug as claimed in claim 7, wherein a half-maximal effective concentration (EC₅₀) of the formula extract is 0.5385 milligrams per milliliter (mg/mL).

9. The drug as claimed in claim 8, wherein the drug comprises a pharmaceutically acceptable carrier.

10. The drug as claimed in claim 9, wherein the pharmaceutically acceptable carrier is selected from the group consisting of powders, granule, tablets, pills, capsules, oral liquids, or ointments.

11. The drug as claimed in claim 10, wherein the drug is used to protect integrity of intestinal barrier of a body; regulate imbalance of intestinal acid-base homeostasis of the body; inhibit death of intestinal epithelial cells of the body; and reduce a level of reactive oxygen species (ROS) in intestinal tract of the body.
